(19) United States
(12) Patent Application Publication
(10) Pub. No.: US 2012/0276571 A1
(43) Pub. Date: Nov. 1, 2012

(54) METHOD OF ANALYZING FORMATION AND PHASE TRANSITION CHARACTERISTIC OF AMORPHOUS CALCIUM CARBONATE

(76) Inventors: Seung Woo Lee, Daejeon (KR); Young Nam Jang, Daejeon (KR)

(21) Appl. No.: 13/359,704

(22) Filed: Jan. 27, 2012

(30) Foreign Application Priority Data
Apr. 29, 2011 (KR) 10-2011-0040481

(51) Int. Cl. G01N 33/50; C12Q 1/02; C12Q 1/25

(52) U.S. Cl. 435/29; 435/4; 436/133

(57) ABSTRACT

Provided is a method of analyzing a formation and a phase transition characteristic of amorphous calcium carbonate that may adjust a preferred orientation in crystalline calcium carbonate as well as an amorphous state of calcium carbonate using a water-soluble material containing an amino acid in an operation of forming calcium carbonate. It is possible to handle issues of a limit of a sampling and a standard pattern of an analysis scheme in in vitro calcium carbonate crystallization test by adjusting a holding time of amorphous calcium carbonate or a preferred orientation of a crystal calcium carbonate when forming calcium carbonate using a water-soluble material containing an amino acid. Further, it is possible to verify further characteristics of elements that adjust a formation of a biological material, which may be used for a synthesis of a new material in tissue engineering as well as for an biomineralizaton process.

### CROSS-REFERENCE TO RELATED APPLICATION

This application claims the benefit of Korean Patent Application No. 10-2011-0040481, filed on Apr. 29, 2011, in the Korean Intellectual Property Office, the disclosure of which is incorporated herein by reference.

### BACKGROUND

1. Field of the Invention

The present invention relates to a method of analyzing a formation and a phase transition characteristic of amorphous calcium carbonate. The present invention relates to a method of analyzing a formation and a phase transition characteristic of amorphous calcium carbonate functioning as a starter in an operation of crystallizing calcium carbonate and a method of analyzing an effect of an organic matter on a formation and a phase transition characteristic of amorphous calcium carbonate, and more particularly, to a method of adjusting an amorphous state of calcium carbonate according to an amount or a type of a water-soluble material containing an amino acid in an operation of forming calcium carbonate, and adjusting a preferred orientation in a crystal phase of calcium carbonate.

2. Description of the Related Art

In addition to metal, a high molecular compound, and the like, a mineral is one of materials widely used in daily life as well as in industrial fields. Among minerals, calcium carbonate (CaCO₃) is one of several abundant minerals existing on Earth, and is a material representative of being synthesized at a relatively low temperature. In particular, CaCO₃ is synthesized from the ocean as well as from the surface of the Earth, and CaCO₃ synthesized from living beings that exist in the ocean, for example, molluscan shell, a tooth of urchin and the like may have biocompatibility when compared to synthesized CaCO₃, and may have an excellent characteristic as a material. CaCO₃ synthesized from the ocean is crystallized in a condition of the ocean corresponding to room temperature and normal pressure and has an excellent configurational characteristic and thus, is expected to be applied to a high value-added business such as a biosensor and tissue engineering as well as to materials industry.

A biological material may refer to a material, for example, a bone, a shell, a skin, and the like synthesized by a living being as well as a human being to survive, and has been commanding attention in chemical and material fields due to an excellent characteristic as a material. Components constituting a biological material synthesized by a living life-form in the ocean may be classified into various biominerals such as a calcium-based biomineral, a silicon-based biomineral, and the like. CaCO₃ is contained in a shell, an exoskeleton of Mollusk or spongy body and the like and thus, may be easily obtained. Further, CaCO₃ has long been used as a research medium due to the excellent characteristic as a material. In particular, research for synthesizing a shell of shellfish may be used as a tool for establishing a mechanism of synthesizing a biological material, and may correspond to an example of verifying biomineralization of a living being.

In nature, CaCO₃ may generally exist in six forms. That is, CaCO₃ may exist as a form of about three polymorphs (vaterite, aragonite and calcite), a form of a hydrate phase containing one or six H₂O, or a form of amorphous CaCo₃.

Among the varied forms of CaCO₃, amorphous CaCO₃ is thermodynamically unstable and thus, easily transitions into a crystal phase under conditions of room temperature and atmospheric pressure. Even though it may be difficult to verify a presence of amorphous CaCO₃ in nature. The presence of amorphous CaCO₃ in shell formation of shellfish could be identified. CaCO₃ constituting a shell of shellfish is known to be in a form of amorphous CaCO₃ at an early stage of spawning, in a form of aragonite at a stage of a larval plankton, and in a form of calcite after a stage of attachment.

However, research on a material in a form of an amorphous phase and a main material controlling the amorphous phase may be associated with a process of synthesizing a biological material by a living being through an interaction of an organic and inorganic mixture. Since the interaction proceeds in a system controlled by a cell or an organic layer, which may be referred to a closed system, and a crystalline size of a synthesized biological material is microscopic. Thus, an accurate verification may not be performed under in vitro environment for a characteristic of amorphous CaCO₃ or an organic material affecting the characteristic of amorphous CaCO₃.

### SUMMARY

An aspect of the present invention provides a method of adjusting an amorphous state of calcium carbonate according to an amount or a type of a water-soluble material containing an amino acid in an operation of forming calcium carbonate, and adjusting a preferred orientation in a crystal phase of calcium carbonate.

According to an aspect of the present invention, there is provided a method of analyzing a formation and a phase transition characteristic of amorphous calcium carbonate, the method including dissolving a water-soluble material in distilled water, forming amorphous calcium carbonate by mixing a calcium agent with the distilled water, and transitioning the amorphous calcium carbonate into crystalline calcium carbonate, wherein an amorphous state of calcium carbonate is adjusted through the water-soluble material.

The water-soluble material may contain an amino acid, and the water-soluble material containing the amino acid may be selected from the group consisting of carbonic anhydrase, bovine serum albumin (BSA), extrapallial fluid (EPF), and hemocyte.

The amino acid may be selected from the group consisting of glycine, alanine, arginine, asparagine, aspartate, cysteine, glutamate, glutamine, histidine, proline, serine, tyrosine, isoleucine, leucine, lysine, tryptophan, valine, methionine, phenylalanine, and threonine.

An amount of the water-soluble material dissolved may be in a range from 0.001 millimolar (mM) to 100 mM.

The calcium agent may be selected from the group consisting of calcium chloride (CaCl₂), calcium sulfate (CaSO₄), calcium bicarbonate (Ca(HCO₃)₂), calcium oxide (CaO), and calcium hydroxide (Ca(OH)₂).

The forming may be performed at a temperature in a range from 5° C. to 45° C.

The forming may be performed at a pressure in a range from 0.1 atmosphere (atm) to 2 atm.

According to an embodiment of the present invention, it is possible to adjust an amorphous state of calcium carbonate using a water-soluble material containing an amino acid in an operation of forming calcium carbonate, and adjust a preferred orientation in a crystal phase of calcium carbonate.

According to another embodiment of the present invention, it is possible to handle issues of a limit of a sampling and a standard pattern of an analysis scheme in in vitro calcium carbonate crystallization test by adjusting a holding time of amorphous calcium carbonate and a preferred orientation in a crystalline calcium carbonate when forming calcium carbonate using a water-soluble material containing an amino acid. Further, it is possible to verify further characteristics of elements that adjust a formation of a biological material, which may be used for a synthesis of a new material in tissue engineering as well as for biomineralization process.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a diagram illustrating a crystalline phase, observed over time using an X-ray diffractometer, of calcium carbonate synthesized when a predetermined amino acid is excluded from distilled water according to a related art;

FIG. 2 is a diagram illustrating a crystalline phase, observed over time using an X-ray diffractometer, of calcium carbonate synthesized when a predetermined amino acid is included in distilled water according to an embodiment of the present invention; and

FIG. 3 is a diagram illustrating a crystalline phase, observed over time using an X-ray diffractometer, of calcium carbonate synthesized when a predetermined amino acid is included in distilled water according to another embodiment of the present invention.

### DETAILED DESCRIPTION

Reference will now be made in detail to exemplary embodiments of the present invention, examples of which are illustrated in the accompanying drawings. Exemplary embodiments are described below to explain the present invention by referring to the figures.

A method of analyzing a formation and a phase transition characteristic of amorphous calcium carbonate may include dissolving a water-soluble material in distilled water, forming amorphous calcium carbonate by mixing a calcium agent with the distilled water, and transitioning the amorphous calcium carbonate into crystalline calcium carbonate, wherein an amorphous state of calcium carbonate is adjusted through the water-soluble material.

In the method of analyzing a formation and a phase transition characteristic of amorphous calcium carbonate, a preferred orientation may be adjusted in the crystalline calcium carbonate through the water-soluble material.

According to embodiments of the present invention, a water-soluble material containing an amino acid may be used to analyze a correlation between calcium carbonate and an organic material. In an operation of forming calcium carbonate, a water-soluble material containing an amino acid may be used to adjust a preferred orientation in crystalline calcium carbonate as well as to adjust an amorphous state of calcium carbonate.

The water-soluble material containing the amino acid may be selected from carbonic anhydrase, bovine serum albumin (BSA), extrapallial fluid (EPF), and hemocyte. The water-soluble material containing the amino acid may contain an amino acid, and may not be limited thereto as long as the water-soluble material may be dissolved in water.

The amino acid may be selected from glycine, alanine, arginine, asparagine, aspartate, cysteine, glutamate, glutamine, histidine, proline, serine, tyrosine, isoleucine, leucine, lysine, tryptophan, valine, methionine, phenylalanine, and threonine.

In the method of analyzing a formation and a phase transition characteristic of amorphous calcium carbonate according to embodiments of the present invention, an amount of the water-soluble material dissolved in distilled water may be in a range from about 0.001 millimolar (mM) to about 100 mM, and more preferably, in a range from about 0.01 mM to about 50 mM.

In the method of analyzing a formation and a phase transition characteristic of amorphous calcium carbonate according to embodiments of the present invention, the calcium agent may be selected from calcium chloride (CaCl₂), calcium sulfate (CaSO₄), calcium bicarbonate (Ca(HCO₃)₂), calcium oxide (CaO), and calcium hydroxide (Ca(OH)₂). Preferably, the calcium agent may be Ca(OH)₂. The calcium agent may not be limited thereto as long as solubility of calcium may be enhanced when mixed with distilled water.

In the method of analyzing a formation and a phase transition characteristic of amorphous calcium carbonate according to embodiments of the present invention, the forming of calcium carbonate by mixing a calcium agent with the distilled water may be performed at a temperature in a range from about 5° C. to about 45° C., and more preferably, in a range from about 10° C. to about 30° C. The forming of calcium carbonate by mixing a calcium agent with the distilled water may be performed at room temperature of 25° C.

In the method of analyzing a formation and a phase transition characteristic of amorphous calcium carbonate according to embodiments of the present invention, the forming of calcium carbonate by mixing a calcium agent with the distilled water may be performed at a pressure in a range from about 0.1 atmosphere (atm) to about 2 atm, and more preferably, in a range from about 0.5 atm to about 1.5 atm. The forming of calcium carbonate by mixing a calcium agent with the distilled water may be performed at a standard pressure of 1 atm.

In the method of analyzing a formation and a phase transition characteristic of amorphous calcium carbonate according to embodiments of the present invention, the forming of calcium carbonate by mixing a calcium agent with the distilled water may be performed at room temperature and standard pressure. As such, the method of analyzing a formation and a phase transition characteristic of amorphous calcium carbonate according to embodiments of the present invention may be performed through a relatively simple operation.

An operation of forming calcium carbonate for a shellfish is as follows. In general, shellfish may use calcium and a carbon ion present in the ocean when synthesizing a shell corresponding to an exoskeleton of the shellfish. In general, shellfish are known to synthesize amorphous calcium carbonate using calcium and a carbon ion, adhere to a supporting body in a form of aragonite at a stage of a larval plankton, and then transition into calcite at a stage of growth, which is illustrated in Reaction mechanism 1.

Ca²⁺+CO₃²⁻→amorphous CaCO₃→CaCO₃ (aragonite)→CaCO₃ (calcite): formation of a shell of a shellfish [Reaction mechanism 1]

An operation of forming calcium carbonate according to embodiments of the present invention may be performed through Reaction mechanism 2 shown below. Using calcium and a carbon ion, amorphous CaCO₃ and CaCO₃.6H₂O may be synthesized, and then may transition into a crystalline phase, that is, calcite.

Ca²⁺+CO₃²⁻→amorphous CaCO₃ & CaCO₃.6H₂O→CaCO₃ (calcite): formation of a CaCO₃ thin film [Reaction mechanism 2]

In this instance, a correlation between calcium carbonate and an organic material may be analyzed using a water-soluble material containing an amino acid. A method according to embodiments of the present invention may use a water-soluble material containing an amino acid to adjust a preferred orientation in crystalline calcium carbonate as well as to adjust an amorphous state of calcium carbonate in a process of forming calcium carbonate. The amorphous state of calcium carbonate and the preferred orientation in crystalline calcium carbonate may be adjusted according to an amount or a type of a predetermined water-soluble material containing an amino acid. In this instance, a predetermined amino acid may correspond to the amino acid described in the foregoing, and an amount of the predetermined amino acid may be in a range from about 0.001 mM to about 100 mM.

Here, adjusting an amorphous state of calcium carbonate may correspond to adjusting a period of time during which calcium carbonate retains an amorphous state. The period of time may correspond to a period of time during which calcium carbonate retains a state of amorphous CaCO₃ & CaCO₃.6H₂O before transitioning from an amorphous state into a crystalline phase as illustrated in Reaction mechanism 2. Thus, an amount or a type of a predetermined water-soluble material containing an amino acid may affect calcium carbonate retaining an amorphous state, and may adjust an amorphous state.

Adjusting a preferred orientation in crystalline calcium carbonate may correspond to changing facial indices of crystalline calcium carbonate when compared to a general calcite. By adjusting a preferred orientation in crystalline calcium carbonate, generated calcium carbonate may be used for various fields such as tissue engineering.

Accordingly, it is possible to handle issues of a limit of a sampling and a standard pattern of an analysis scheme in an in vitro calcium carbonate crystallization test by adjusting a holding time of amorphous calcium carbonate or a crystalline phase of calcium carbonate when forming calcium carbonate using a water-soluble material containing an amino acid according to embodiments of the present invention. Further, it is possible to verify further characteristics of elements that adjust a formation of a biological material, which may be used for a synthesis of a new material in tissue engineering as well as for an operation of a biomineralization.

Hereinafter, embodiments of the present invention will be described with reference to drawings. In a method of analyzing a formation and a phase transition characteristic of amorphous calcium carbonate according to embodiments of the present invention, Ca(OH)₂ corresponding to a calcium agent may be mixed with distilled water, in which a predetermined amount of amino acid is dissolved, corresponding to a water-soluble material, and then a phase transition characteristic of calcium carbonate synthesized on a surface of distilled water and a morphological characteristic of a calcium carbonate thin film are observed.

After mixing about 50 grams (g) of Ca(OH)₂ with about one liter (L) of distilled water in which from about 5 to about 25 mM of serine and arginine are dissolved to synthesize calcium carbonate at room temperature and standard pressure, calcium carbonate formed according to a predetermined period of time is analyzed by separately collecting a number of 100 millileter (mL) of samples. An X-ray diffractometer may be used to analyze a crystalline phase.

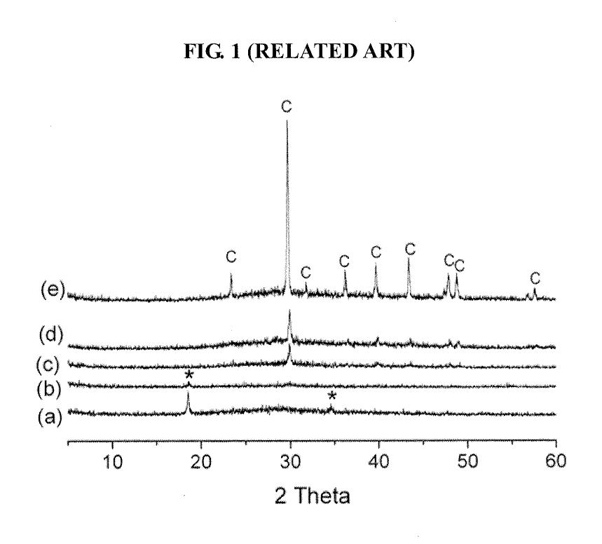

FIG. 1 is a diagram illustrating a crystalline phase, observed over time using an X-ray diffractometer, of calcium carbonate synthesized when a predetermined amino acid is excluded from distilled water according to a related art.

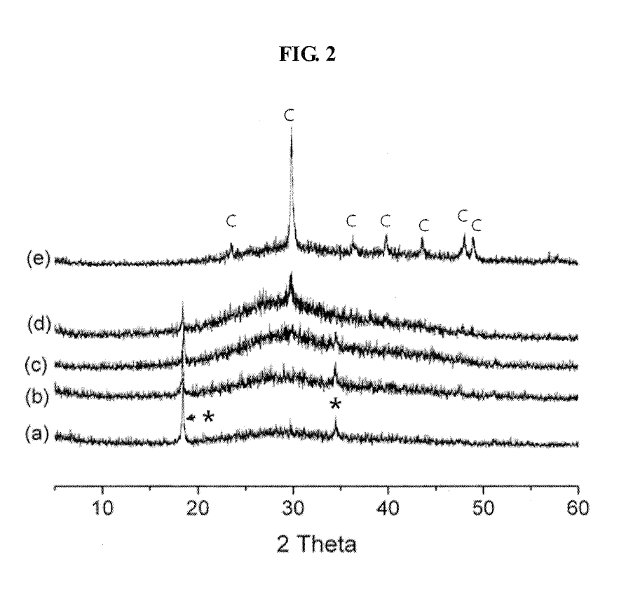

FIG. 2 is a diagram illustrating a crystalline phase, observed over time using an X-ray diffractometer, of calcium carbonate synthesized when a predetermined amino acid is included in distilled water according to an embodiment of the present invention.

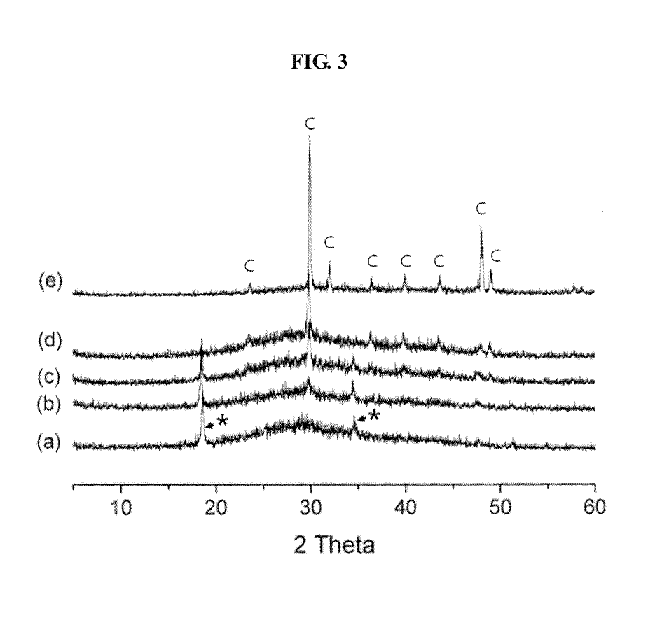

FIG. 3 is a diagram illustrating a crystalline phase, observed over time using an X-ray diffractometer, of calcium carbonate synthesized when a predetermined amino acid is included in distilled water according to another embodiment of the present invention.

A difference in a crystalline phase of calcium carbonate may be verified among FIG. 1 corresponding to a crystalline phase of calcium carbonate excluding a predetermined amino acid, FIG. 2 corresponding to a crystalline phase of calcium carbonate including serine, and FIG. 3 corresponding to a crystalline phase of calcium carbonate including arginine.

Each reading (a) through (e) of FIG. 1 denotes a passage of time after Ca(OH)₂ is mixed with distilled water excluding an amino acid. Here, (a) corresponds to 10 minutes, (b) corresponds to 30 minutes, (c) corresponds to one hour, (d) corresponds to two hours, and (e) corresponds to 24 hours.

Each reading (a) through (e) of FIG. 2 denotes a passage of time after Ca(OH)₂ is mixed with distilled water including 25 mM of serine. Here, (a) corresponds to 10 minutes, (b) corresponds to 30 minutes, (c) corresponds to one hour, (d) corresponds to two hours, and (e) corresponds to 24 hours.

Each of (a) through (e) of FIG. 3 denotes a passage of time after Ca(OH)₂ is mixed with distilled water including 25 mM of arginine. Here, (a) corresponds to 10 minutes, (b) corresponds to 30 minutes, (c) corresponds to one hour, (d) corresponds to two hours, and (e) corresponds to 24 hours.

A characteristic of amorphous calcium carbonate may be provided in a shape of a knoll having a convex portion in a middle corresponding to a 2 theta (θ) value of about 20 to about 35 degrees.

Referring to FIG. 1, when an amino acid is excluded, a time during which CaCO₃ and CaCO₃.6H₂O retains an amorphous form is relatively short, and a characteristic of CaCO₃.6H₂O ceases to exist within 30 minutes of a reaction holding time. Referring to FIG. 2 and FIG. 3, when an amino acid is included, a time during which CaCO₃ and CaCO₃.6H₂O retains an amorphous form is relatively long.

FIG. 2 illustrates that CaCO₃.6H₂O exists after two hours of retaining an amorphous form in the atmosphere when serine is included, and FIG. 3 illustrates that CaCO₃.6H₂O exists after one hour of retaining an amorphous form in the atmosphere when arginine is included.

The existence of CaCO₃.6H₂O is indicated by “*” in FIG. 1 through FIG. 3. In the embodiments of the present invention, a silicon (Si)-low background sample holder may be used to eliminate an amorphous phase effect of a glass holder. When an amino acid such as serine and arginine is included, amorphous calcium carbonate may remain in an amorphous phase for a relatively long period of time when compared to amorphous calcium carbonate excluding an amino acid.

A peak intensity of a crystalline phase of calcite may vary depending on a type of an amino acid, as illustrated in Table 1. Figures in Table 1 indicate degrees of preferred orientations, and are based on index (104). As illustrated in Table 1, preferred orientations of CaCO₃ crystalline phases may be different from each other when an amino acid is dissolved.

TABLE 1
Relative proportion of preferred orientation

| | | 25 mM of | 25 mM of | |
|---|---|---|---|---|
| | | serine is | arginine is | |
| | amino acid is | dissolved as | dissolved as | |
| facial indices | excluded | amino acid | amino acid | general calcite |
| 012 | 12.3 | 9.3 | 5.2 | 12 |
| 104 | 100 | 100 | 100 | 100 |
| 006 | 3.2 | 1.5 | 11.2 | 3 |
| 110 | 11.5 | 7.7 | 2.9 | 14 |
| 113 | 19.8 | 15.3 | 8.6 | 18 |
| 202 | 23.6 | 11.6 | 13.0 | 18 |
| 018 | 24.7 | 27.2 | 47.2 | 17 |
| 116 | 15.8 | 16.8 | 19.0 | 17 |
| 211 | 2.4 | 1.8 | 5.6 | 4 |
| 122 | 5.9 | 5.6 | 2.6 | 8 |

Referring to Table 1, comparing to a case in which an amino acid is excluded and a case in which arginine is dissolved, dissolving serine may not help develop a predetermined surface. When arginine is dissolved, indices (006), (018), and (116) may be relatively more developed. In particular, index (006), corresponding to a coordinate surface of index (001), may be a crystal face interrelated with a growth of shellfish.

The test result shows an effect of dissolving arginine on enhancement of a preferred orientation of index (001). A preferred orientation according to a type of an amino acid may function as an element affecting a morphological characteristic of a CaCO₃ thin film. A crystalline phase of a synthesized CaCO₃ thin film may be different from a crystalline phase of calcite corresponding to a thermodynamically stable form of polymorphism of typical calcium carbonate, which may result from an effect of a CaCO₃ thin film having a bladelike shape.

Accordingly, a method of analyzing a formation and a phase transition characteristic of amorphous calcium carbonate may adjust a preferred orientation in crystalline calcium carbonate as well as an amorphous state of calcium carbonate. A water-soluble material containing a predetermined amino acid such as serine and arginine may affect a control of a shape or a time during which CaCO₃ retains an amorphous form when forming calcium carbonate.

It may be possible to handle issues of a limit of a sampling and a standard pattern of an analysis scheme in an in vitro calcium carbonate crystallization test by adjusting a holding time of amorphous calcium carbonate or a preferred orientation of a crystal calcium carbonate when forming calcium carbonate using a predetermined amino acid according to embodiments of the present invention. Further, it may be possible to verify further characteristics of elements that adjust a formation of a biological material, which may be used for a synthesis of a new material in tissue engineering as well as for an operation of a biomineralization.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A method of analyzing a formation and a phase transition characteristic of amorphous calcium carbonate, the method comprising:
dissolving a water-soluble material in distilled water;
forming amorphous calcium carbonate by mixing a calcium agent with the distilled water; and
transitioning the amorphous calcium carbonate into crystalline calcium carbonate,
wherein an amorphous state of calcium carbonate is adjusted through the water-soluble material.

2. The method of claim 1, wherein:
the water-soluble material contains an amino acid, and the water-soluble material containing the amino acid is selected from the group consisting of carbonic anhydrase, bovine serum albumin (BSA), extrapallial fluid (EPF), and hemocyte.

3. The method of claim 2, wherein the amino acid is selected from the group consisting of glycine, alanine, arginine, asparagine, aspartate, cysteine, glutamate, glutamine, histidine, praline, serine, tyrosine, isoleucine, leucine, lysine, tryptophan, valine, methionine, phenylalanine, and threonine.

4. The method of claim 1, wherein an amount of the water-soluble material dissolved is in a range from 0.001 millimolar (mM) to 100 mM.

5. The method of claim 1, wherein the calcium agent is selected from the group consisting of calcium chloride (CaCl₂), calcium sulfate (CaSO₄), calcium bicarbonate (Ca(HCO₃)₂), calcium oxide (CaO), and calcium hydroxide (Ca(OH)₂).

6. The method of claim 1, wherein the forming is performed at a temperature in a range from 5° C. to 45° C.

7. The method of claim 1, wherein the forming is performed at a pressure in a range from 0.1 atmosphere (atm) to 2 atm.
